(19) United States
(12) Patent Application Publication
(10) Pub. No.: US 2014/0180072 A1
(43) Pub. Date: Jun. 26, 2014

(54) System and Method for Precisely Locating an Intravascular Device

(71) Applicant: Volcano Corporation, San Diego, CA (US)

(72) Inventors: Justin Davies, London (GB); Jerry Litzza, Sacramento, CA (US)

(21) Appl. No.: 14/137,349

(22) Filed: Dec. 20, 2013

Related U.S. Application Data
(60) Provisional application No. 61/745,507, filed on Dec. 21, 2012.

(51) Int. Cl. A61B 6/12; A61B 8/08

(52) U.S. Cl. CPC A61B 6/12; A61B 8/0841
USPC 600/424

(57) ABSTRACT

Systems and methods for locating invasive intravascular devices within a vascular system are provided. In one embodiment, an invasive medical sensing system is disclosed. The system comprises a flexible elongate member having a plurality of radiation-sensitive components arranged around an outer circumferential surface of the flexible elongate member. The plurality of radiation-sensitive components is arranged such that an orientation of the flexible elongate member can be determined when the sensors are exposed to radiation produced by a radiation source. The system further comprises a watchdog component communicatively coupled to the plurality of radiation-sensitive components and operable to detect radiation-induced changes in behavior of the plurality of radiation-sensitive components caused by the radiation and to determine the orientation of the flexible elongate member relative to the radiation source based on the detected radiation-induced changes in behavior.

### CROSS-REFERENCE TO RELATED APPLICATION

The present application claims the benefit of the filing date of provisional U.S. Patent Application No. 61/745,507 filed Dec. 21, 2012. The entire disclosure of this provisional application is incorporated herein by this reference.

### TECHNICAL FIELD

The present disclosure relates generally to intravascular medical diagnosis and treatment and, in particular, to X-ray location of invasive intravascular devices.

### BACKGROUND

Innovations in diagnosing and verifying the level of success of treatment of disease have migrated from external imaging processes to internal diagnostic processes. In particular, diagnostic equipment and processes have been developed for diagnosing vasculature blockages and other vasculature disease by means of ultra-miniature sensors placed upon the distal portion of a flexible elongate member such as a catheter, guide catheter, or a guide wire used for catheterization procedures. For example, known medical sensing techniques include angiography, intravascular ultrasound (IVUS), forward looking IVUS (FL-IVUS), fractional flow reserve (FFR) determination, coronary flow reserve (CFR) determination, optical coherence tomography (OCT), trans-esophageal echocardiography, and image-guided therapy. Each of these techniques may be better suited for different diagnostic situations. To increase the chance of successful treatment, health care facilities may have a multitude of imaging, treatment, diagnostic, and sensing modalities on hand in a catheter lab during a procedure. Similarly, intravascular devices are also commonplace in therapeutic procedures. In a variety of treatments protocols, a flexible elongate member is advanced through the vasculature to the site of dysfunction. These intravascular treatments include balloon angioplasty, vascular stenting, valve repair, valve replacement, rotational atherectomy, and intravascular ablation including RF ablation and ultrasound ablation.

While existing invasive intravascular devices have proved useful, they have not been entirely satisfactory in all respects. One particular challenge involves determining the precise location of the elongate member within the patient. The inclusion of radiographic fiducials provides an adequate method of locating the device in general. However, location using fiducials is imprecise, subjective, and is limited by the two-dimensional nature of the radiographic image. Accordingly, the need exists for improved devices and methods for pinpoint location of invasive intravascular devices.

### SUMMARY

Embodiments of the present disclosure provide a system and method for precisely and objectively determining the location of an invasive intravascular device using penetrating energy.

The systems and methods of the present disclosure utilize radiation-sensitive circuits disposed at the distal portion of an elongate member to determine an orientation of the elongate member relative to a source of penetrating energy such as an X-ray emitter, gamma ray emitter, and/or other energy source. The radiation-sensitive circuits are monitored and radiation intensity is determined from the effect on the circuits' behavior. The orientation of the elongate member can then be determined from the intensity of the radiation as measured by the circuits. This provides an accurate and objective method for determining position, especially compared to systems and methods that rely on a human operator to interpret a radiographic image.

In some embodiments, an invasive medical sensing system is provided. The system comprises a flexible elongate member having a plurality of radiation-sensitive components arranged around an outer circumferential surface of the flexible elongate member such that an orientation of the flexible elongate member can be determined when the sensors are exposed to radiation produced by a radiation source. The system further comprises a watchdog component communicatively coupled to the plurality of radiation-sensitive components and operable to detect radiation-induced changes in behavior of the plurality of radiation-sensitive components caused by the radiation and to determine the orientation of the flexible elongate member relative to the radiation source based on the detected radiation-induced changes in behavior. In one such embodiment, the elongate member further includes a sensor corresponding to a medical sensing modality disposed along a distal portion of the elongate member. At least one component of the plurality of radiation-sensitive components is physically incorporated into the sensor.

In some embodiments, an intravascular ultrasound system is provided. The system comprises a flexible elongate member having an ultrasound transducer system disposed at a distal portion of the flexible elongate member, where the ultrasound transducer system includes a plurality of radiation-sensitive components arranged around an outer circumferential surface of the flexible elongate member. The intravascular ultrasound system further comprises a patient-interface monitor communicatively coupled to the ultrasound transducer system via the flexible elongate member, a processing system communicatively coupled to the ultrasound transducer system via the patient-interface monitor, and a watchdog component communicatively coupled to the plurality of radiation-sensitive components. The watchdog component is operable to detect radiation-induced changes in behavior of the plurality of radiation-sensitive components caused by radiation produced by a radiation source and to determine an orientation of the flexible elongate member relative to the radiation source based on the detected radiation-induced changes in the behavior of the plurality of radiation-sensitive components.

In some embodiments, a method of locating a flexible elongate member within a vessel is provided. The method comprises advancing the flexible elongate member having a plurality of radiation-sensitive components disposed at a distal portion of the flexible elongate member into the vessel. The plurality of radiation-sensitive components is exposed to penetrating energy generated by an energy source. An operational behavior of the plurality of radiation-sensitive components is measured while the components are exposed to the penetrating energy. Based on the measured operational behavior, an orientation of the flexible elongate member relative to the energy source is determined.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIGS. 1A, 1B, and 1C are schematic drawings depicting an invasive intravascular system in various applications according to some embodiments of the present disclosure. In particular, FIG. 1A is illustrative of the intravascular system according to some embodiments of the present disclosure. FIG. 1B is illustrative of the intravascular system in a cardiac catheterization procedure according to some embodiments of the present disclosure. FIG. 1C is illustrative of the intravascular system in a renal catheterization procedure according to some embodiments of the present disclosure.

FIG. 2 is a cross-sectional view of a portion of a flexible elongate member according to some embodiments of the present disclosure.

FIG. 3 is a cross-sectional view of a portion of a flexible elongate member according to some embodiments of the present disclosure.

FIG. 4 is a simplified schematic illustration of a solid-state ultrasound transducer system according to some embodiments of the present disclosure.

FIG. 5 is a top view of a portion of an ultrasound transducer system depicted in its flat form according to some embodiments of the present disclosure.

FIG. 6 is a cross-sectional view of a control region of an ultrasound system depicted in its rolled form according to some embodiments of the present disclosure.

FIG. 7 is a diagram of an exemplary user interface for presenting orientation information according to some embodiments of the multi-modality processing system.

FIG. 8 is a flow diagram of a method of determining an orientation of a flexible elongate member according to some embodiments of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the invasive intravascular system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

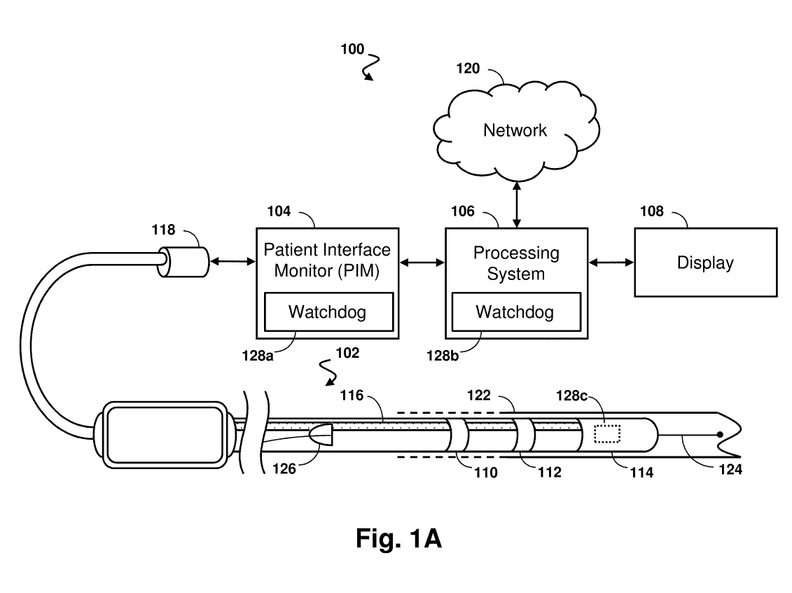

FIGS. 1A, 1B, and 1C are schematic drawings depicting an invasive intravascular system 100 in various applications according to some embodiments of the present disclosure. In particular, FIG. 1A is illustrative of the intravascular system 100 according to some embodiments of the present disclosure. FIG. 1B is illustrative of the intravascular system 100 in a cardiac catheterization procedure according to some embodiments of the present disclosure. FIG. 1C is illustrative of the intravascular system 100 in a renal catheterization procedure according to some embodiments of the present disclosure. It is understood that these procedures are merely exemplary, and the system 100 is suitable for use in any type of vasculature including peripheral, intracranial, cardiac, renal, lymphatic, and other vascular systems.

With reference to FIG. 1A, the invasive intravascular system 100 may be a single modality medical system or a multi-modality medical system. In that regard, a multi-modality medical system provides for coherent integration and consolidation of multiple forms of acquisition and processing elements designed to be sensitive to a variety of methods used to acquire and interpret human biological physiology and morphological information and/or coordinate treatment of various conditions. The invasive intravascular system 100 may be used to perform on a patient any number of medical sensing procedures such as angiography, intravascular ultrasound (IVUS), photoacoustic IVUS, forward looking IVUS (FL-IVUS), virtual histology (VH), intravascular photoacoustic (IVPA) imaging, pressure determination, fractional flow reserve (FFR) determination, coronary flow reserve (CFR) determination, optical coherence tomography (OCT), computed tomography, intracardiac echocardiography (ICE), forward-looking ICE (FLICE), intravascular palpography, transesophageal ultrasound, or any other medical sensing modalities known in the art. The invasive intravascular system 100 may also be used to perform on a patient any number of therapeutic procedures such as balloon angioplasty, vascular stenting, rotational atherectomy, radio-frequency ablation, and ultrasound ablation.

In one embodiment, the system 100 includes a computer system with the hardware and software to acquire, process, and display medical imaging data, but, in other embodiments, the system 100 includes any other type of computing system operable to process medical data. In the embodiments in which the system 100 includes a computer workstation, the system includes a processor such as a microcontroller or a dedicated central processing unit (CPU), a non-transitory computer-readable storage medium such as a hard drive, random access memory (RAM), and/or compact disk read only memory (CD-ROM), a video controller such as a graphics processing unit (GPU), and/or a network communication device such as an Ethernet controller and/or wireless communication controller. In that regard, in some particular instances, the system 100 is programmed to execute steps associated with the data acquisition and analysis described herein. Accordingly, it is understood that any steps related to data acquisition, data processing, instrument control, and/or other processing or control aspects of the present disclosure may be implemented by the system 100 using corresponding instructions stored on or in a non-transitory computer readable medium accessible by the processing system. In some instances, the system 100 is portable (e.g., handheld, on a rolling cart, etc.). Further, it is understood that in some instances system 100 comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure.

The invasive intravascular system 100 includes a flexible elongate member 102, a patient interface module (PIM) 104, a processing system 106, and/or a display 108. The flexible elongate member 102 carries one or more sensors (e.g., sensors 110, 112, and 114) disposed at the distal portion of the elongate member 102. For clarity, only three sensors are illustrated, although the present principles may be extended to systems incorporating any number of sensors, including 1, 2, 4, 8, 16, and 24 sensor embodiments. In various embodiments, sensors, including sensors 110, 112, and 114, correspond to sensing modalities such as flow volume, IVUS, photoacoustic IVUS, FL-IVUS, pressure, fractional flow reserve (FFR) determination, coronary flow reserve (CFR) determination, OCT, transesophageal echocardiography, image-guided therapy, other suitable modalities, and/or combinations thereof. In an exemplary embodiment, sensors 110, 112, and 114 include IVUS ultrasound transceivers. In a further exemplary embodiment, sensor 114 includes an IVUS ultrasound transceiver and sensors 110 and 112 include pressure sensors. In yet another exemplary embodiment, sensor 114 includes an FL-IVUS transceiver. Other embodiments incorporate other combinations of sensors, and no particular sensor or combination of sensors is required for any particular embodiment. The flexible elongate member may also include a connecting conduit 116 that carries data between the sensors in the distal portion of the elongate member 102 and a coupler 118 at the proximal end. The connecting conduit 116 may include an optical fiber, a stranded conductor bundle, and/or another suitable connecting device, and in some embodiments, takes the form of a wireless connection such as IEEE 802.11 Wi-Fi standards, Ultra Wide-Band (UWB) standards, wireless FireWire, wireless USB, or another high-speed wireless networking standard.

The sensors 110, 112, and 114, the connecting conduit 116, and other associated components of the flexible elongate member 102 are sized and shaped to allow for the diameter of the elongate member 102 to be very small. In various examples, the outside diameter of the elongate member 102, such as a guide wire, guide catheter, or catheter, containing one or more electronic, optical, and/or electro-optical components as described herein is between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members 102 incorporating the electronic, optical, and/or electro-optical component(s) of the present application are suitable for use in a wide variety of lumens within a human patient besides those that are part of or immediately surround the heart, including veins and arteries of the extremities, renal arteries, blood vessels in and around the brain, and other lumens.

At a high level, the elongate member 102 physically supports the sensors 110, 112, and 114 as they are navigated through the vasculature and communicatively couples the sensors to the PIM 104 via the connector 118. In turn, the patient interface module (or PIM) 104 facilitates communication of signals between the processing system 106 and the elongate member 102. This may include generating control signals that configure the sensors of the elongate member 102, supplying power to operate the sensors, and/or transferring data measurements captured by the sensors to the processing system 106. In one embodiment, the PIM 104 includes analog to digital (A/D) converters and transmits digital sensor data to the processing system 106. In other embodiments, the PIM 104 transmits analog data to the processing system 106. In one embodiment, the PIM 104 transmits the medical sensing data over a Peripheral Component Interconnect Express (PCIe) data bus connection, but, in other embodiments, it may transmit data over a USB connection, a Thunderbolt connection, a FireWire connection, or some other high-speed data bus connection. In other instances, the PIM 104 is connected to the processing system 106 via wireless connections using IEEE 802.11 Wi-Fi standards, Ultra Wide-Band (UWB) standards, wireless FireWire, wireless USB, or another high-speed wireless networking standard. In some embodiments, the PIM 104 performs preliminary signal processing prior to transmitting the signals to the processing system 106. In examples of such embodiments, the PIM 104 performs amplification, filtering, and/or aggregating of the data.

The processing system 106 receives the sensing data from the elongate member 102 by way of the PIM 104 and processes the data for viewing on the display 108. In embodiments incorporating visualizing modalities, this may include creating an image of the tissues surrounding the elongate member 102. The processing system 106 may also store and transmit both raw and processed sensor data to other systems and devices. In that regard, the processing system 106 may be communicatively coupled to a data network 120. In the illustrated embodiment, the data network 120 is a TCP/IP-based local area network (LAN); however, in other embodiments, it may utilize a different protocol such as Synchronous Optical Networking (SONET), or may be a wide area network (WAN). The processing system 106 may connect to various resources via the network 120. For example, the processing system 106 may communicate with a Digital Imaging and Communications in Medicine (DICOM) system, a Picture Archiving and Communication System (PACS), and/or a Hospital Information System (HIS) through the network 120. Additionally, in some embodiments, a network console may communicate with the processing system 106 via the network 120 to allow a doctor or other health professional to access the aspects of the invasive intravascular system 100 remotely. For instance, a user of the network console may access patient medical data such as diagnostic images collected by multi-modality processing system 106, or, in some embodiments, may monitor or control one or more on-going procedures in the catheter lab in real-time. The network console may be any sort of computing device with a network connection such as a PC, laptop, smartphone, tablet computer, or other such device located inside or outside of a health care facility.

The flexible elongate member 102 is sized and structured to be passed into a vessel 122 for purposes of measuring the surrounding environment. Vessel 120 represents fluid filled or surrounded structures, both natural and man-made, within a living body that may be measured or sensed and can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. In addition to sensing natural structures, the sensed structures may also include man-made structures such as, but without limitation, heart valves, stents, shunts, filters and other devices positioned within the body. Accordingly, the flexible elongate member 102 may take the form of a catheter, a guide wire, and/or a guide catheter designed for intravascular use. In some embodiments, a separate guide wire 124 is first inserted into the vessel 122, and the flexible elongate member 102 is advanced over top the guide wire 124. Accordingly, in some embodiments, the flexible elongate member is a rapid-exchange catheter and includes a guide wire exit port 126 that allows the guided wire to be threaded through a lumen of the elongate member 102 in order to direct the elongate member 102 through the vessel 122.

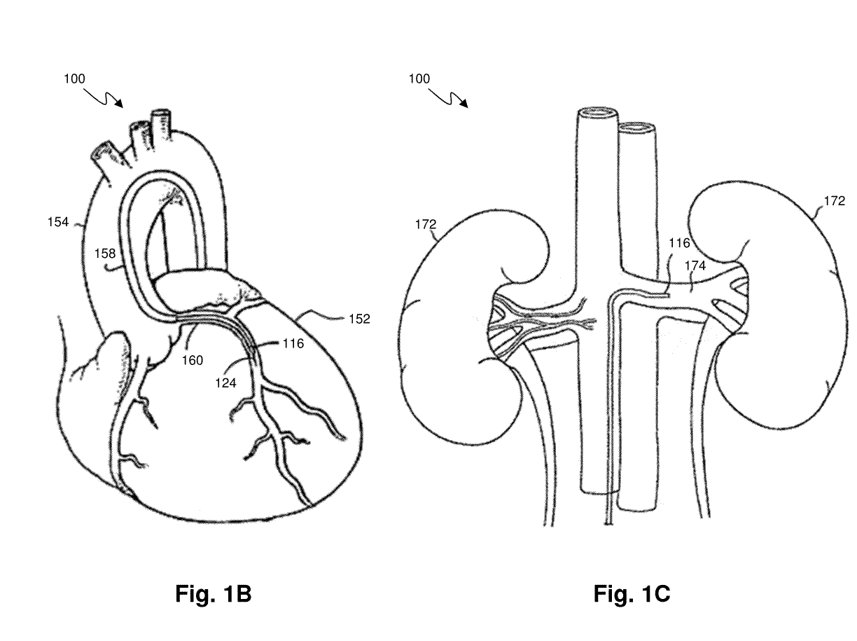

One or more of the PIM 104, the processing system 106, and/or the elongate member 102 includes a watchdog monitor (e.g., watchdogs 128a, 128b, and 128c) which monitors the behavior of radiation-sensitive circuits disposed within the elongate member 102 in order to determine the orientation of the elongate member 102 relative to a radiation source. The operation of the watchdog is disclosed in detail below with respect to FIG. 2-8.

With reference now to FIG. 1B, an application of the invasive intravascular system 100 includes a coronary catheterization procedure. In a coronary catheterization procedure, the elongate member 102 is passed into a blood vessel of the heart 152 via the aorta 154. In some embodiments, a guide wire 124 is first advanced into the heart 152 through a large peripheral artery leading into the aorta 154. Once the guide wire 124 is properly located, a guide catheter 158 is advanced over the guide wire. The elongate member 102 is then directed into place by traveling over the guide wire 124 and inside the guide catheter 158. In further embodiments, the elongate member 102 is advanced without a guide catheter and/or guide wire. In the illustrated embodiment, the distal portion of the elongate member 102 is advanced until it is positioned in the left coronary artery 160. Sensors disposed within the elongate member 102 are activated, and the sensing data is passed along the elongate member 102 to components of the system 100 such as the PIM 104 and/or the processing system 106 of FIG. 1A. In the example of an elongate member 102 incorporating IVUS sensors, signals sent from the PIM 104 to one or more ultrasound transducers of the elongate member 102 cause the transducers to emit a specified ultrasonic waveform. Portions of the ultrasonic waveform are reflected by the surrounding vasculature and received by one or more receiving transducers. The resulting echo signals are amplified within the elongate member 102 for transmission to the PIM 104. In some instances, the PIM 104 amplifies the echo data, performs preliminary pre-processing of the echo data, and/or retransmits the echo data to the processing system 106. The processing system 106 then aggregates and assembles the received echo data to create an image of the vasculature for display.

In some exemplary applications, the elongate member 102 is advanced beyond the area of the vascular structure to be measured and pulled back as the sensors are operating, thereby exposing a longitudinal portion of the vessel. To ensure a constant velocity, a pullback mechanism is used in some applications. A typical withdraw velocity is 0.5 mm/s, although other rates are possible based on beam geometry, sample speed, and the processing power of the system. In some embodiments, the elongate member 102 includes an inflatable balloon portion. As part of a treatment procedure, the device may be positioned adjacent to a stenosis (narrow segment) or an obstructing plaque within the vascular structure and inflated in an attempt to widen the restricted area.

With reference now to FIG. 1C, another application of the invasive intravascular system 100 includes a renal catheterization procedure. In a renal catheterization procedure, the elongate member 102 is passed into a blood vessel of the kidneys 172 via the aorta. This may involve first advancing a guide wire and/or guide catheter and using the guide device(s) to control the advance of the elongate member 102. In the illustrated embodiment, the distal portion of the elongate member 102 is advanced until it is located in the right renal artery 174. Then, the elongate member 102 is activated and signals are passed between the elongate member 102 and components of the system 100 such as the PIM 104 and/or the processing system 106 of FIG. 1A. The structures of the renal vasculature differ from those of the cardiac vasculature. Vessel diameters, tissue types, and other differences may mean that operating parameters suited to cardiac catheterization are less well suited to renal catheterization and vice versa. Furthermore, renal catheterization may target different structures, seeking to image the renal adventitia rather than arterial plaques, for example. For these reasons and more, the invasive intravascular system 100 may support different operating parameters for different applications such as cardiac and renal imaging. Likewise, the concept may be applied to any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body.

Referring now to FIG. 2, illustrated is a cross-sectional view of a flexible elongate member 200. The elongate member 200 is suitable for use with the invasive intravascular system 100 of FIG. 1A and may be substantially similar to elongate member 102 of FIG. 1A. In various embodiments, the flexible elongate member 200 includes sensors (not illustrated) corresponding to sensing modalities such as flow volume, IVUS, photoacoustic IVUS, FL-IVUS, pressure, fractional flow reserve (FFR) determination, coronary flow reserve (CFR) determination, OCT, transesophageal echocardiography, image-guided therapy, other suitable modalities, and/or combinations thereof. The flexible elongate member 200 may also include a connecting conduit 202 that carries data from the sensors in the distal portion of the elongate member 102 to a connecting coupler at the proximal end and a lumen 210 such as a guide wire lumen. The flexible elongate member 200 may take the form of a guide wire, a catheter, or a guide catheter and is sized for intravascular use. Thus, the outside diameter of the elongate member 200 as described herein is between about 0.0007″ (0.0178 mm) and about 0.118″ (3.0 mm), with some particular embodiments having outer diameters of approximately 0.014″ (0.3556 mm) and approximately 0.018″ (0.4572 mm)).

The flexible elongate member 200 also includes radiation-sensitive circuits (including circuits 204a, 204b, and 204c) disposed around the circumference of the member 200 and used to determine the orientation of the flexible elongate member 200 within the patient. The radiation-sensitive circuits exhibit a change in an electrical property when exposed to types of penetrating radiation including X-rays, gamma radiation, electron beams, alpha radiation, beta radiation, neutron beams, and/or other types of radiation. A watchdog (not shown) located in the elongate member 200 and/or a device communicatively coupled to the elongate member 200, such as a PIM or a processing system, monitors the radiation-sensitive circuits for radiation-induced changes in behavior. When a radiation source (indicated by arrow 206) is directed towards the elongate member 200, the watchdog determines the orientation of the elongate member 200 relative to the radiation source based on the magnitude of the effect produced at each of the radiation-sensitive circuit. The flexible elongate member 200 of FIG. 2 includes three radiation-sensitive circuits 204a, 204b, and 204c disposed around the circumferential area of the member 200. However, the concepts of the present disclosure can be extended to elongate members with any number of circuits. For example, embodiments incorporate 2, 3, 4, 5, 7, 8, 16, 32 and more radiation-sensitive circuits.

In the example, an external X-ray source is directed at the patient from a direction indicated by arrow 206. The emitted radiation penetrates the surrounding tissue and exposes the radiation-sensitive circuits 204a, 204b, and 204c. Circuits directly exposed to the radiation (circuits 204a and 204b in the illustrated embodiment) have a stronger response than circuits shielded from the radiation by the elongate member 200 (circuit 204c in the illustrated embodiment). To create a larger differential, the elongate member 200 may include a radiopaque material 208 that blocks a significant portion of the radiation. Radiopacity is typically a function of electron density, and a number of radiopaque materials suitable for use in vivo are known in the art. In that regard, the radiopaque material 208 may include heavy metals, ceramic materials, and/or high-density thermoplastics. In some embodiments, a guide wire (not shown) passing through a lumen 210 of the elongate member 200 blocks a portion of the radiation further contributing to the radiation differential. By analyzing the response of the radiation-sensitive circuits across the circuits, the watchdog can determine an orientation (e.g., an orientation in relation to axes 212x, 212y, and/or 212z) of the elongate member relative to the radiation source. In some embodiments, the raw measurements of the circuits are compared to each other to determine the circuits most closely aligned with the radiation source. In further embodiments, the raw measurements are converted into radiation intensity values prior to the comparison. To account for variations in the circuits and their associated sensitivities, a baseline of measure of operation may be established for each circuit in the absence of the radiation source or with the radiation source turned off. Subsequent measurements may be compared to the baseline to determine the intensity of exposure more accurately.

In an exemplary embodiment, the radiation-sensitive circuits (e.g., circuits 204a, 204b, and 204c) include X-ray photodiodes, such as CMOS silicon photodiodes. Photodiodes can be operated in a photovoltaic mode where the output voltage of the photodiode is proportional to the intensity of the radiation, or photoconductive mode where the conductance of the photodiode is proportional to the intensity of the radiation. In either mode, the photodiodes are capable of not only detecting radiation but also gauging the intensity. In further embodiments, the radiation-sensitive circuits include charged coupled devices (CCDs), active photosensors, and/or other photosensors known to one of skill in the art. Furthermore, general-purpose semiconductor devices tend to have radiation-sensitive behavior. For example, radiation may generate additional free electrons in the semiconductor leading to an increase in band gap noise. This noise can be monitored to determine the relative dose of radiation received by the device. Therefore, in some embodiments, a component of a sensor (e.g., a flow sensor, a pressure sensor, an IVUS transducer, an FL-IVUS transducer, an OCT transceiver, etc.) in the elongate member 200 is also used as a radiation-sensitive circuit.

To further enhanced accuracy, some embodiments incorporate directional radiation-sensitive circuits. Directionally-focused circuits exhibit reduced sensitivity to radiation directed at oblique angles. In some such embodiments, the directionally-focused circuits are arranged such that they are most sensitive along a radial axis substantially perpendicular to the circumferential surface of the elongate member 200 (i.e., axis 214 of sensors 204a). This configuration may produce in a greater variation in radiation measurement of sensors on the portion of the circumferential surface unshielded from the radiation source by the elongate member 200.

In some applications, X-ray fluoroscopy used to image the elongate member 200 and obtain a general location is also used to determine an orientation utilizing the radiation-sensitive circuits. Obtaining a general location and a fine-grained orientation concurrently using the same source may be more-efficient than a two-stage process and reduces the exposure dosage of the patient. In some applications, therapeutic radiation such as a radiosurgical treatment used in a therapeutic capacity incidentally exposes the radiation-sensitive circuits and is used to determine an orientation of the elongate member 200 relative to the treated area.

Utilizing radiation-sensitive circuits allows operators to determine the orientation of the elongate member 200 quickly and reliably. Orientation is particularly important for devices with a directional bias (e.g., a side port, a side-looking sensor, a side-firing ablation element, a preformed permanent bend, etc.). Orientation is also particularly important when device is symmetrical, but the sensor data is directional. For example, when presented with IVUS data indicating an arterial plaque, it may be important to determine the exact location of the plaque along the vessel wall. For these reasons and others, the ability to obtain a precise determination of the orientation of the flexible elongate member 200 allows a surgeon to guide the elongate member 200 through complex vasculature, to better image the surrounding vasculature, and to deliver targeted treatments more effectively.

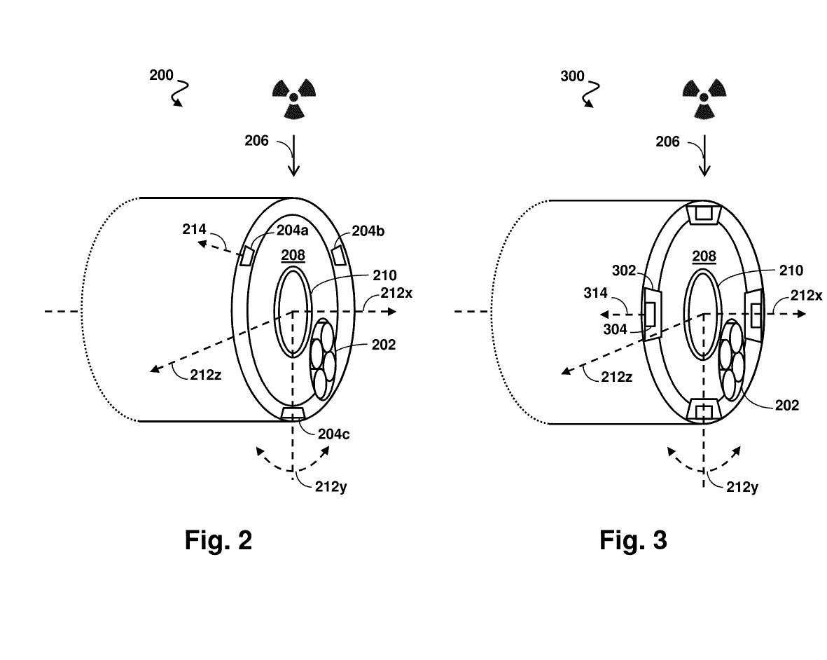

FIG. 3 is a cross-sectional view of a flexible elongate member 300 according to some embodiments of the present disclosure. The elongate member 300 is suitable for use with the invasive intravascular system 100 of FIG. 1A and may be substantially similar to elongate member 102 of FIG. 1A and elongate member 200 of FIG. 2. In various embodiments, the flexible elongate member 300 includes sensors 302 corresponding to sensing modalities such as flow volume, IVUS, photoacoustic IVUS, FL-IVUS, pressure, fractional flow reserve (FFR) determination, coronary flow reserve (CFR) determination, OCT, transesophageal echocardiography, image-guided therapy, other suitable modalities, and/or combinations thereof. The flexible elongate member 300 may also include a connecting conduit 202 that carries data from the sensors in the distal portion of the elongate member 300 to a connecting coupler at the proximal end and may include a lumen 210 such as a guide wire lumen. The flexible elongate member 300 may take the form of a guide wire, a catheter, or a guide catheter and is sized for intravascular use. Thus, the outside diameter of the elongate member 300 as described herein may be between about 0.0007″ (0.0178 mm) and about 0.118″ (3.0 mm), with some particular embodiments having outer diameters of approximately 0.014″ (0.3556 mm) and approximately 0.018″ (0.4572 mm)).

In contrast to the elongate member 200 of FIG. 2, the flexible elongate member 300 includes four radiation-sensitive circuits 304 used to determine the orientation of the flexible elongate member 300 in the patient, although the number of radiation-sensitive circuits is not limiting. The radiation-sensitive circuits 304 exhibit a change in an electrical property in the presence of penetrating radiation including X-rays, gamma radiation, electron beams, alpha radiation, beta radiation, neutron beams, and other types of emissions. Therefore, when a radiation source (arrow 206), representing a known reference point, is directed towards the elongate member 300, the effect on the radiation-sensitive circuits 304 is used to measure the orientation of the elongate member 300 relative to the reference point. For example, the radiation sensitive circuits 304 may be used to measure an orientation in relation to axes 212x, 212y, and/or 212z. Exemplary radiation-sensitive circuits 304 include X-ray photodiodes, CCDs, active photosensors, and/or other photosensors known to one of skill in the art.

In the illustrated embodiment, the radiation-sensitive circuits 304 are incorporated into the sensors 302 of the elongate member 300. The radiation-sensitive circuits 304 may be a discrete circuit element and/or a part of a functional of component of the sensor 302 known to have a measurable sensitivity to penetrating radiation. In some embodiments, a remaining portion of the sensor 302 and/or the sensor packaging partially shields the circuit 304 from radiation directed at oblique angles and leaves an unshielded portion of the circuit 304 directed towards the outer circumference of the elongate member 300 as indicated by axis 314. This increases the directional sensitivity of the circuit 304 and, in many such embodiments, increases the accuracy of the orientation determination. In some such embodiments, the sensor 302 and/or the sensor packaging is specially adapted to provide shielding from penetrating radiation. For example, the sensor packaging material may include heavy metals, ceramics, and/or high-density plastics. Similar to the embodiments of FIG. 2, the radiation-sensitive circuits 304 may be directionally-focused circuits and may be aligned with the greatest sensitivity directed along the axis 314 substantially perpendicular to the circumferential surface of the elongate member 300. Also similar to the embodiments of FIG. 2, the elongate member 300 may include a radiopaque material 208 that blocks a significant portion of the radiation directed at the member 300 and that shields circuits 304 on the circumferential surface directed away from the radiation source. The radiopaque material may include heavy metals, ceramic materials, and/or high-density thermoplastics. In some embodiments, a guide wire (not shown) passing through a lumen 210 of the elongate member 300 blocks a portion of the radiation further contributing to a radiation differential.

When an external X-ray source is directed at the patient, (e.g., the source indicated by arrow 206), the energy exposes the radiation-sensitive circuits 304. Circuits directly exposed to the radiation have a stronger response than circuits shielded from the radiation by the elongate member 300. By analyzing the response of the radiation-sensitive circuits across the circuits, the orientation (e.g., relative to axes 212x, 212y, and/or 212z) of the elongate member relative to the radiation source can be determined. Incorporating the radiation-sensitive circuits 304 into the sensors 302, whether as dedicated single-purpose components or utilizing other functional components of the sensor 302, may allow reuse of existing sensor circuitry such as differential comparators, amplifiers, analog-to-digital converters, interface circuitry and/or other circuitry. This may result in a smaller (reduced diameter), more maneuverable, elongate member 300.

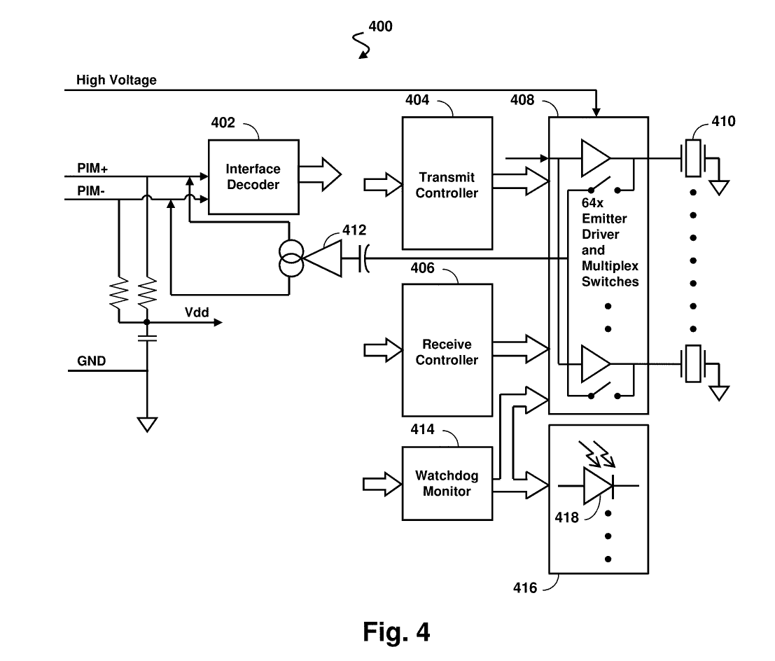

FIG. 4 is a simplified schematic illustration of a solid-state ultrasound transducer system 400 according to some embodiments of the present disclosure. Transducer system 400 is merely one non-limiting example of a sensor incorporating radiation-sensitive circuitry suitable for use in an elongate member such as elongate member 102 of FIG. 1A, elongate member 200 of FIG. 2, and/or elongate member 300 of FIG. 3. The transducer system 400 may be a piezoelectric micromachine ultrasound transducer (PMUT) system, a capacitive micromachined ultrasound transducer (CMUT) system, a piezoelectric transducer (PZT) system, and/or any combination thereof. U.S. Pat. No. 6,238,347, entitled “ULTRASONIC TRANSDUCER ARRAY AND METHOD OF MANUFACTURING THE SAME,” U.S. Pat. No. 6,641,540, entitled “MINIATURE ULTRASOUND TRANSDUCER,” U.S. Pat. No. 7,226,417, entitled “HIGH RESOLUTION INTRAVASCULAR ULTRASOUND TRANSDUCER ASSEMBLY HAVING A FLEXIBLE SUBSTRATE,” and U.S. Pat. No. 7,914,458, entitled “CAPACITIVE MICROFABRICATED ULTRASOUND TRANSDUCER-BASED INTRAVASCULAR ULTRASOUND PROBES,” disclose IVUS transducer systems in more detail and are herein incorporated by reference. Examples of commercially available products that include suitable IVUS transducers include, without limitation, the Eagle Eye® series of IVUS catheters, the Revolution® IVUS catheter, and the Visions® series of IVUS catheters, each available from Volcano Corporation.

The system 400 includes seven major blocks, the interface decoder 402, the transmit controller 404, the receive controller 406, the driver and multiplexer array 408, the ultrasound transducers 410, the echo amplifier 412, and the watchdog 414. In physical implementations, any of the major blocks of the system 400 may be divided among one or more separate integrated circuit chips.

At a high level, sets of ultrasound transducers 410 are selected to send ultrasonic energy and capture reflected ultrasonic echoes. The echo data is amplified and transmitted back to a processing system (e.g., processing system 106 of FIG. 1) via a PIM (e.g., PIM 104 of FIG. 1). The system 400 includes a data bus for receiving signals that control the ultrasound emission and echo capture and for transmitting captured echo data. In the illustrated embodiment, the data bus is a differential pair, PIM+ and PIM−, of bidirectional multipurpose signals. In some implementations, the PIM+/− signal pair is used to: (1) supply low-voltage DC power (V_{dd}) to drive the circuitry of the system 400, (2) operate as a serial communication channel to permit the configuration of the transmit controller 404, the receive controller 406, the multiplexer array 408, and/or the watchdog 414, (3) operate as a serial communication channel to support advanced features such as programmability and status reporting, (4) carry the transmit trigger pulses as a balanced differential signal from a PIM to activate the transmitter and timing circuitry, and (5) conduct the balanced output signal from the echo amplifier 412 to the PIM.

The interface decoder 402 converts PIM+/− instruction into control signals for the remaining components of the system 400. These control signals may include configuration information and transmit triggers. Configuration information may be used by the transmit controller 404 to select one or more transmitting transducers 410 and by the receive controller 406 to select one or more receiving transducers 410. The transmit 404 and receive 406 controllers select the appropriate transducers using the multiplexer array 410. When a transmit trigger is received, drivers within the multiplexer array 410 cause the selected emitting transducer(s) 410 to produce an ultrasonic waveform. The waveform is reflected by the tissue and other structures near and around the transducer 410 creating ultrasonic echoes that are captured by the receiving transducer(s) selected by the multiplexer array 410. The received echo signal may be boosted by an echo amplifier 412. In the illustrated embodiment, the echo amplifier 412 is a differential amplifier, although other amplifier types are contemplated. The amplified signal is then transmitted over the data bus.

In the illustrated embodiment, the system 400 also includes a watchdog 414 that monitors a set of radiation-sensitive circuits to determine the amount of penetrating radiation received by each circuit. From this information, the orientation of an elongate member containing the system 400 can be determined relative to the radiation source. In the illustrated embodiment, the radiation-sensitive circuits include an array 416 of photodiodes 418, although in alternate embodiments, the radiation-sensitive circuits may be CCDs, active photosensors, and/or other photosensitive circuits known to one of skill in the art. As an alternative to discrete photodetectors, the radiation-sensitive circuits may be circuits within other functional blocks that exhibit changes in behavior when exposed to penetrating radiation. For example, in some embodiments, the watchdog 414 monitors circuits of the multiplexer array 408 for changes in operation attributable to penetrating radiation such as an increase in band gap noise. By monitoring the operation of the radiation-sensitive circuits, the relative orientation of the elongate member containing the system 400 may be determined.

In the illustrated embodiment, the exposure data is transmitted by the watchdog 414 to the PIM via the PIM+/− signal pair. In further embodiments, the exposure data is transmitted over an alternate channel including a wireless communication channel. In some embodiments, the watchdog 414 is external to the flexible elongate member that contains the radiation-sensitive circuits. In such embodiments, the watchdog 414 may be implemented within the PIM and/or the processing system.

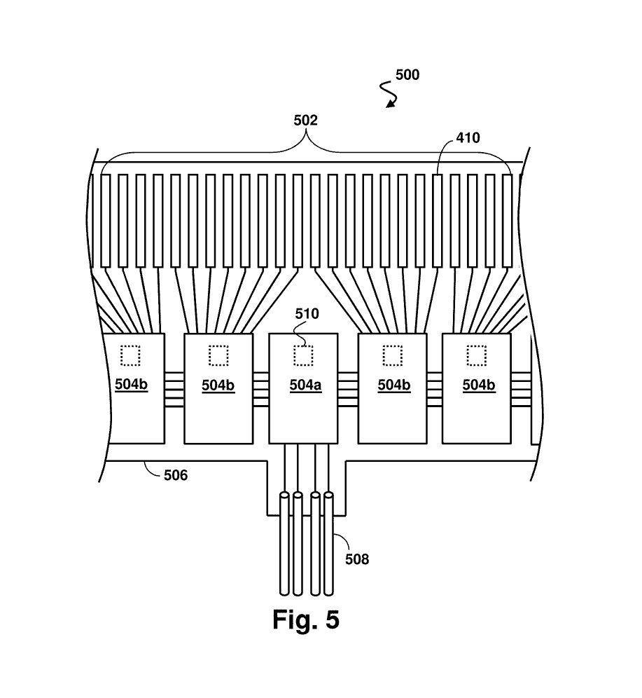

FIG. 5 is a top view of a portion of an ultrasound transducer system 500 depicted in its flat form according to some embodiments of the present disclosure. In many embodiments, the system 500 is partially assembled in the flat form and subsequently shaped into a rolled form during final assembly. The system 500 includes a transducer array 502 and transducer control circuits (including controllers 504a and 504b) attached to a flex circuit 506. As indicated by the common reference numbers, the ultrasound transducers 410 of the transducer array 502 may be substantially similar to those disclosed with reference to FIG. 4. The transducer array 502 may include any number and type of ultrasound transducers 410, although for clarity only a limited number of ultrasound transducers are illustrated in FIG. 5. In an embodiment, the transducer array 502 includes 64 individual ultrasound transducers 410. In a further embodiment, the transducer array 502 includes 32 ultrasound transducers. Other numbers of transducers are both contemplated and provided for. In an embodiment, the ultrasound transducers 410 of the transducer array 502 are piezoelectric micromachined ultrasound transducers (PMUTs) fabricated on a microelectromechanical system (MEMS) substrate using a polymer piezoelectric material. In alternate embodiments, the transducer array includes piezoelectric zirconate transducers (PZT) transducers such as bulk PZT transducers, capacitive micromachined ultrasound transducers (cMUTs), single crystal piezoelectric materials, other suitable ultrasound transmitters and receivers, and/or combinations thereof.

In the illustrated embodiment, the system 500 having 64 ultrasound transducers 410 includes nine transducer control circuits (including control circuits 504a and 504b), of which five are shown. Designs incorporating other numbers of transducer control circuits including 8, 9, 16, 17 and more are utilized in other embodiments. In some embodiments, a single controller is designated a master controller and configured to receive signals directly from a cable 508. The remaining controllers are slave controllers. In the depicted embodiment, the master controller 504a does not directly control any transducers 410. In other embodiments, the master controller 504a drives the same number of transducers 410 as the slave controllers 504b or drives a reduced set of transducers 410 as compared to the slave controllers 504b. In the illustrated embodiment, a single master controller 504a and eight slave controllers 504b are provided. Eight transducers are assigned to each slave controller 504b. Such controllers may be referred to as 8-channel controllers based on the number of transducers they are capable of driving.

One or more of the controllers may include radiation-sensitive circuits (indicated by outlines 510) designed to assist in determining an orientation of the system 500 relative to a radiation source. The radiation-sensitive circuits 510 are substantially similar to those disclosed with respect to FIGS. 1-4. In some embodiments, the radiation-sensitive circuits 510 are discrete circuits such as photodiodes, CCDs, active photosensors, and/or other photosensitive circuits known to one of skill in the art. In some embodiments, the radiation-sensitive circuits 510 are functional circuits that perform various tasks involved in the generation and collection of ultrasound data and that also exhibit a change in operation when exposed to penetrating radiation such as an increase in band gap noise.

The control circuits 504a and 504b are attached to a flex circuit 506. The flex circuit 506 provides structural support and physically connects the transducer control circuits 504a and/or 504b to the transducers 410. The flex circuit 506 may contain a film layer of a flexible polyimide material such as KAPTON™ (trademark of DuPont). Other suitable materials include polyester films, polyimide films, polyethylene napthalate films, or polyetherimide films, other flexible printed circuit substrates as well as products such as Upilex® (registered trademark of Ube Industries) and TEFLON® (registered trademark of E.I. du Pont). The film layer is configured to be wrapped around a ferrule to form a cylindrical toroid in some instances. Therefore, the thickness of the film layer is generally related to the degree of curvature in the final assembled system 500. In some embodiments, the film layer is between 5 μm and 100 μm, with some particular embodiments being between 12.7 μm and 25.1 μm.

In an embodiment, the flex circuit 506 further includes conductive traces formed on the film layer. Conductive traces carry signals between the transducer control circuits 504a and/or 504b and the transducers 410 and provide a set of pads for connecting the conductors of cable 508. Suitable materials for the conductive traces include copper, gold, aluminum, silver, tantalum, nickel, and tin and may be deposited on the flex circuit 506 by processes such as sputtering, plating, and etching. In an embodiment, the flex circuit 506 includes a chromium adhesion layer. The width and thickness of the conductive traces are selected to provide proper conductivity and resilience when the flex circuit 506 is rolled. In that regard, an exemplary range for the thickness of a conductive trace is between 10-50 μm. For example, in an embodiment, 20 μm conductive traces are separated by 20 μm of space. The width of a conductive trace may be further determined by the size of a pad of a device or the width of a wire to be coupled to the trace.

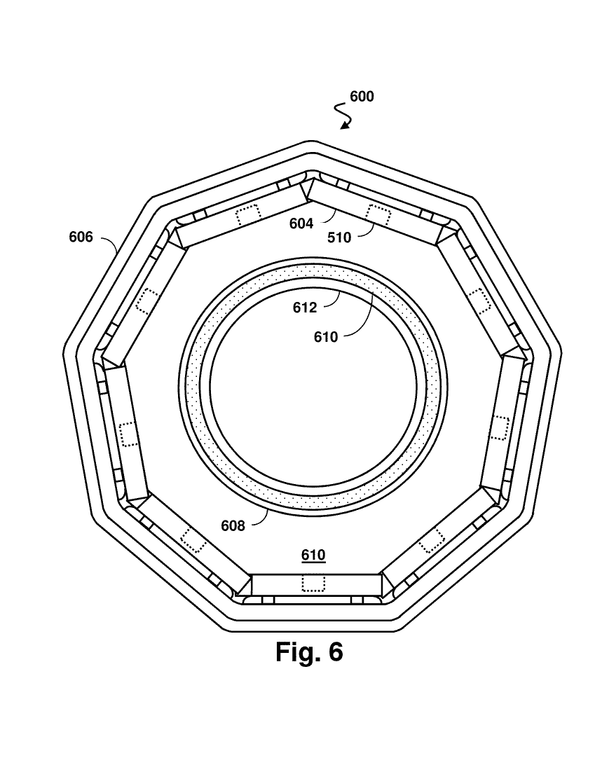

FIG. 6 is a cross-sectional view of a control region 600 of an ultrasound system depicted in its rolled form according to some embodiments of the present disclosure. The control region 600 contains transducer control circuits 604 substantially similar to transducer control circuits 504a and 504b of FIG. 5. In that regard, the transducer control circuits contain radiation-sensitive circuits 510 that are substantially similar to those disclosed with respect to FIGS. 1-5. In some embodiments, the radiation-sensitive circuits 510 are discrete circuits such as photodiodes, CCDs, active photosensors, and/or other photosensitive circuits known to one of skill in the art. In some embodiments, the radiation-sensitive circuits 510 are functional circuits that perform various tasks involved in the generation and collection of ultrasound data and that exhibit a change in operation when exposed to penetrating radiation such as an increase in band gap noise.

The transducer control circuits 604 are bonded to a flex circuit 506 substantially similar to that of FIG. 5. In some embodiments, the control region 600 includes a retaining structure 608 applied over the transducer control circuits 604. The retaining structure 608 may be used during the rolling process, for example, to secure components including the control circuits 604. The retaining structure 608 may also include a radiopaque material to partially shield the radiation-sensitive circuits 510 in the rolled form. Encapsulating epoxy 610 fills the space between the transducer control circuits 604 and the retaining structure 608 and/or between the retaining structure 608 and a ferrule 612 in some embodiments. Similar to the retaining structure 608, the encapsulating epoxy 610 may also include a radiopaque material to partially shield the radiation-sensitive circuits 510.

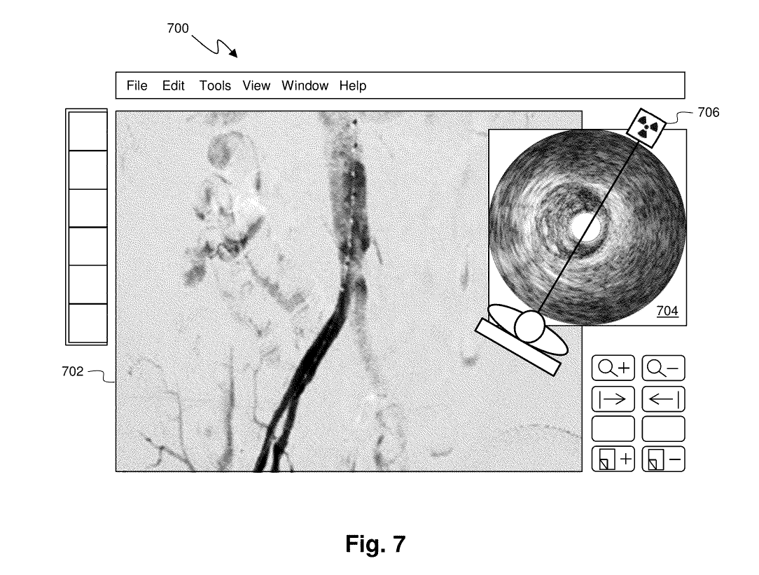

FIG. 7 is a diagram of an exemplary user interface 700 for presenting orientation information according to some embodiments of the multi-modality processing system. The user interface 700 represents one possible arrangement for displaying the information presented by the invasive intravascular system 100 of FIGS. 1A, 1B, and 1C. One skilled in the art will recognize that alternate arrangements are both contemplated and provided for.

In the illustrated embodiment, the user interface 700 includes two data display panes 702 and 704 presenting data corresponding to two different sensing modalities. Further embodiments include other numbers of display panes and likewise present other numbers of modalities. Display pane 702 presents a fluoroscopic image of the patient with an angiographic projection. In some embodiments, a radiocontrast agent is introduced into the relevant vasculature to enhance the contrast of the vessels. Radiographic fiducials disposed along an elongate member allow the operator to discern the general location of the elongate member from the fluoroscopic image. However, in the illustrated embodiment, it is particularly difficult to discern the orientation of the elongate member from the fluoroscopic image. Instead, the orientation can be determined using a number of radiation-sensitive circuits according to the principles of the present disclosure. In the illustrated embodiment, the radiation-sensitive circuits are incorporated into one or more IVUS transducers contained within the distal portion of the elongate member. Display pane 704 presents IVUS data collected by the IVUS transducer(s) of the elongate member and also presents an orientation marker 706 that depicts the orientation of the elongate member relative to a radiation source, which in this embodiment, is the radiation source used to generate the fluoroscopic image shown in display pane 702. In the illustrated embodiment, the IVUS data maintains a fixed alignment, and the orientation marker 706 is rotated around the IVUS data to indicate relative position. In further embodiments, the orientation marker 706 is fixed and the IVUS data is rotated to indicate relative position. In further embodiments, both the orientation marker 706 and the IVUS data may be rotated independently. In still a further configuration, a 3-dimensional graphical representation of the sensing element is displayed on the screen and oriented such that it matches the orientation in relation to the radiation source. The 3-dimensional representation may be co-registered with the fluoroscopic image.

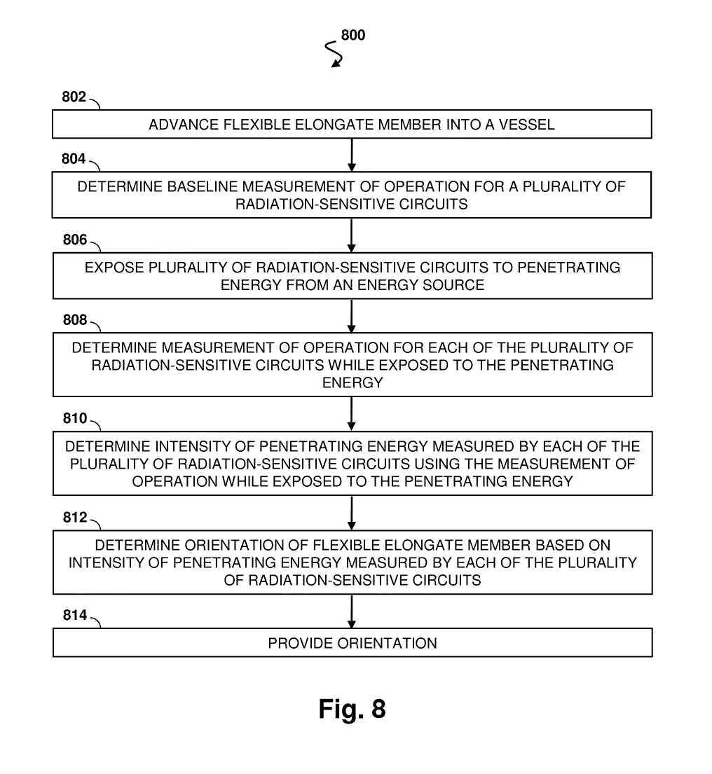

FIG. 8 is a flow diagram of a method 800 of determining an orientation of a flexible elongate member according to some embodiments of the present disclosure. It is understood that additional steps can be provided before, during, and after the steps of method 800 and that some of the steps described can be replaced or eliminated for other embodiments of the method. The method 800 may be performed by a watchdog (e.g., watchdogs 128a, 128b, and 128c of FIG. 1A, watchdog 414 of FIG. 4, etc.) in order to determine an orientation of the flexible elongate member relative to a radiation source. In block 802, the flexible elongate member, which may take the form of a catheter, a guide catheter, a guide wire, and/or other invasive intravascular device, is advanced into a vessel. The vessel represents fluid filled or surrounded structures, both natural and man-made, within a living body and can include for example, but without limitation, structures such as: organs including the liver, heart, kidneys, gall bladder, pancreas, lungs; ducts; intestines; nervous system structures including the brain, dural sac, spinal cord and peripheral nerves; the urinary tract; as well as valves within the blood or other systems of the body. The elongate member includes a plurality of radiation-sensitive circuits arranged around an outer circumferential surface of the elongate member. The elongate member may also include one or more sensors corresponding to one or more medical sensing modalities, such as flow volume, IVUS, photoacoustic IVUS, FL-IVUS, pressure, fractional flow reserve (FFR) determination, coronary flow reserve (CFR) determination, OCT, transesophageal echocardiography, image-guided therapy, other suitable modalities, and/or combinations thereof.

In block 804, a first or baseline measurement of operation for each of the plurality of radiation-sensitive circuits is established in the absence of a radiation source, or with the radiation source turned off. In block 806, the radiation source exposes the elongate member and the radiation-sensitive circuits with a penetrating energy such as an X-ray emission, a gamma ray emission, an electron beam, alpha radiation, beta radiation, a neutron beam, and/or other types of penetrating energy known to one of skill in the art. In block 808, a second measurement of operation is taken for each of the plurality of radiation-sensitive circuits while exposed to the penetrating energy. In block 810, the second measurement of operation is used to determine the intensity of the penetrating energy measured at each of the plurality of radiation-sensitive circuits. This may include converting the raw measurement of operation into a measure of radiation intensity or dose. In block 812, the orientation of the flexible elongate member relative to the radiation source is determined from the measurements of the plurality of radiation-sensitive circuits. In some embodiments, the measurements are compared across the plurality of circuits to determine the degree to which the respective circuits were shielded by an interposed portion of the elongate member. Likewise, in some embodiments, the relative radiation intensities are compared across the plurality of directionally-focused circuits to determine the angle at which the respective circuits are oriented to the radiation source. In block 814, the orientation is provided and may be presented to a user through an image on a display, such as a graphical representation. In one form, the graphical representation of the orientation of the sensing device is superimposed on an X-ray or fluoroscopic image. In another form, the graphical representation is spaced apart from the X-ray or fluoroscopic image on the display; although the graphical representation and the image(s) may be co-registered.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An invasive medical sensing system comprising:
a flexible elongate member having a plurality of radiation-sensitive components arranged around an outer circumferential surface of the flexible elongate member, wherein the plurality of radiation-sensitive components is arranged such that an orientation of the flexible elongate member can be determined when the sensors are exposed to radiation produced by a radiation source; and
a watchdog component communicatively coupled to the plurality of radiation-sensitive components and operable to:
detect radiation-induced changes in behavior of the plurality of radiation-sensitive components caused by the radiation; and
determine the orientation of the flexible elongate member relative to the radiation source based on the detected radiation-induced changes in behavior.

2. The system of claim 1, wherein the watchdog component is further operable to:
determine a baseline behavior of the plurality of radiation-sensitive components in the absence of the radiation; and
compare the detected radiation-induced changes in behavior to the baseline behavior,
wherein the determining of the orientation of the flexible elongate member relative to the radiation source is further based on the comparison of the detected radiation-induced changes in behavior to the baseline behavior.

3. The system of claim 1, wherein the watchdog component is further operable to determine the intensity of the radiation received at each of the plurality of radiation-sensitive components based on the detected radiation-induced changes in behavior,
wherein the determining of the orientation of the flexible elongate member relative to the radiation source is further based on the determined intensity of the radiation received at each of the plurality of radiation-sensitive components.

4. The system of claim 1, wherein the watchdog component is further operable to compare the radiation-induced changes in behavior across the plurality of radiation-sensitive components,
wherein the determining of the orientation of the flexible elongate member relative to the radiation source is further based on the comparison of the radiation-induced changes in behavior across the plurality of radiation-sensitive components.

5. The system of claim 1, wherein the elongate member includes a sensor disposed along a distal portion of the elongate member, the sensor corresponding to a medical sensing modality, wherein at least one component of the plurality of radiation-sensitive components is physically incorporated into the sensor.

6. The system of claim 5, wherein the at least one component of the plurality of radiation-sensitive components further performs a sensing function related to the medical sensing modality.

7. The system of claim 1, wherein the flexible elongate member further includes a radiopaque core.

8. The system of claim 1, wherein at least one component of the plurality of radiation-sensitive components is directionally focused and exhibits reduced sensitivity to radiation directed oblique to an axis.

9. The system of claim 1, wherein the axis is substantially perpendicular to the outer circumferential surface of the flexible elongate member.

10. The system of claim 1, wherein the radiation is one of an X-ray emission, a gamma ray emission, an electron beam, alpha radiation, beta radiation, and a neutron beam.

11. An intravascular ultrasound system comprising:
a flexible elongate member having an ultrasound transducer system disposed at a distal portion of the flexible elongate member, the ultrasound transducer system including a plurality of radiation-sensitive components arranged around an outer circumferential surface of the flexible elongate member;
a patient-interface monitor communicatively coupled to the ultrasound transducer system via the flexible elongate member;
a processing system communicatively coupled to the ultrasound transducer system via the patient-interface monitor; and
a watchdog component communicatively coupled to the plurality of radiation-sensitive components and operable to:
detect radiation-induced changes in behavior of the plurality of radiation-sensitive components caused by radiation produced by a radiation source; and
determine an orientation of the flexible elongate member relative to the radiation source based on the detected radiation-induced changes in the behavior of the plurality of radiation-sensitive components.

12. The system of claim 11, wherein the watchdog component is physically located within at least one of the flexible elongate member, the patient-interface monitor, and the processing system.

13. The system of claim 11, wherein the plurality of radiation-sensitive components is physically located within a plurality of ultrasound transducer controllers of the ultrasound transducer system.

14. The system of claim 13, wherein the plurality of radiation-sensitive components includes an array of photodiodes.

15. The system of claim 13, wherein the plurality of radiation-sensitive components includes an ultrasound transducer multiplexer of the ultrasound transducer system.

16. The system of claim 11, wherein the watchdog component is further operable to:
determine a baseline behavior of the plurality of radiation-sensitive components in the absence of the radiation; and
compare the detected radiation-induced changes in behavior to the baseline behavior,
wherein the determining of the orientation of the flexible elongate member relative to the radiation source is further based on the comparison of the detected radiation-induced changes in behavior to the baseline behavior.

17. The system of claim 11, wherein the watchdog component is further operable to determine the intensity of the radiation received at each of the plurality of radiation-sensitive components based on the detected radiation-induced changes in behavior,
wherein the determining of the orientation of the flexible elongate member relative to the radiation source is further based on the determined intensity of the radiation received at each of the plurality of radiation-sensitive components.

18. The system of claim 11, wherein the watchdog component is further operable to compare the radiation-induced changes in behavior across the plurality of radiation-sensitive components,
wherein the determining of the orientation of the flexible elongate member relative to the radiation source is further based on the comparison of the radiation-induced changes in behavior across the plurality of radiation-sensitive components.

19. The system of claim 11, wherein the flexible elongate member further includes a radiopaque core.

20. The system of claim 11, wherein the radiation is one of an X-ray emission, a gamma ray emission, an electron beam, alpha radiation, beta radiation, and a neutron beam.

21. A method of locating a flexible elongate member within a vessel comprising:
advancing the flexible elongate member into the vessel, the flexible elongate member having a plurality of radiation-sensitive components disposed at a distal portion of the flexible elongate member;
exposing the plurality of radiation-sensitive components to penetrating energy generated by an energy source;
measuring an operational behavior of the plurality of radiation-sensitive components while exposed to the penetrating energy;
determining an orientation of the flexible elongate member relative to the energy source based on the measured operational behavior.

22. The method of claim 21 further comprising:
determining a baseline measurement of operation for the plurality of radiation-sensitive components in the absence of the penetrating energy; and
comparing the measured operational behavior to the baseline measurement,
wherein the determining of the orientation of the flexible elongate member relative to the energy source is further based on the comparison of the measured operational behavior to the baseline measurement.

23. The method of claim 21 further comprising:
determining an intensity of exposure for each component of the plurality of radiation-sensitive components based on the measured operational behavior,
wherein the determining of the orientation of the flexible elongate member relative to the energy source is further based on the determined intensity of exposure for each component.

24. The method of claim 21 further comprising:
comparing the measured operational behavior of the plurality of radiation-sensitive components while exposed to the penetrating energy across the plurality of radiation-sensitive components,
wherein the determining of the orientation of the flexible elongate member relative to the energy source is further based on the comparison of the measured operational behavior of the plurality of radiation-sensitive components while exposed to the penetrating energy across the plurality of radiation-sensitive components.
